(19) United States
(12) Patent Application Publication
(10) Pub. No.: US 2019/0076095 A1
(43) Pub. Date: Mar. 14, 2019

(54) TREATMENT REGIMEN COMPLIANCE MODIFICATION SYSTEM

(71) Applicant: CHILDREN'S HOSPITAL AND CLINICS OF MINNESOTA, Minneapolis, MN (US)

(72) Inventors: Laura Marie GANDRUD PICKETT, Edina, MN (US); Jeffrey WENESS, Shakopee, MN (US)

(21) Appl. No.: 15/771,796

(22) PCT Filed: Oct. 28, 2016
(86) PCT No.: PCT/US2016/059384

§ 371 Date: Apr. 27, 2018

Related U.S. Application Data
(60) Provisional application No. 62/248,451, filed on Oct. 30, 2015.

(51) Int. Cl. A61B 5/00; A61B 5/145; G16H 40/67; G16H 10/60

(52) U.S. Cl. CPC A61B 5/6898; A61B 5/14532; G06F 2221/2149; A61B 5/0022; G16H 10/60; G16H 40/67

(57) ABSTRACT

A treatment regimen compliance modification system includes devices for modifying compliance with a treatment regimen by a subject having a chronic health condition. The devices include a mechanism for locking down a subject's computing device if the subject has not complied with the treatment regimen. When the subject is determined to have not complied with the treatment regimen, the subject's computing device can be at least partially shut down and then automatically released after a predetermined amount of time.

This application is being filed on 28 Oct. 2016, as a PCT International patent application, and claims priority to U.S. Provisional Patent Application No. 62/248,451, filed Oct. 30, 2015, the disclosure of which is hereby incorporated by reference herein in its entirety.

### BACKGROUND

Chronic life impacting conditions, such as chronic diseases, are long lasting health conditions that can be controlled but not cured. Examples of chronic life impacting conditions include diabetes, epilepsy, asthma, allergy, heart disease, Alzheimer's disease, breast cancer, glaucoma, and obesity. Although such chronic conditions are costly health problems, the chronic conditions are preventable or can be effectively controlled in various manners.

In certain cases, people who suffer from chronic diseases are provided with prescribed treatment regimens designed to manage or improve the chronic diseases. Such treatment regimens may require patients to take actions on a regular basis. For example, some treatment regimens for managing the effects of diabetes provide for monitoring a diabetic's blood glucose level by using a blood glucose meter to periodically obtain a blood glucose reading from the diabetic's blood. Such treatment regimens may include a schedule by which blood glucose readings should be taken, and/or an acceptable range within which a blood glucose level should fall. In order for treatment regimens to provide effective guidance to diabetics for managing his or her blood glucose levels, it is important for diabetics to strictly comply with the schedule so that their blood glucose levels are properly monitored.

To adhere to the testing schedules, diabetics typically use portable blood glucose meters to measure blood glucose levels throughout a given period of time, such as a day or a week. Using a blood glucose meter may help diabetics better manage their diabetes by tracking changes over time in their blood glucose levels. However, a process of using a blood glucose meter periodically in accordance with a prescribed schedule may be tedious to some diabetics, in particular children or teenagers, who may not fully appreciate either the serious nature of their conditions or the importance of regular blood glucose readings. A failure to test blood glucose levels or comply with treatment regimens can potentially cause the diabetics to experience serious medical complications.

### SUMMARY

In general terms, this disclosure is directed to a system and method for modifying a subject's compliance with a treatment regimen configured for managing the subject's chronic health condition. In one possible configuration and by non-limiting example, the system and method provide a disincentive device for encouraging the subject to comply with the treatment regimen. Various aspects are described in this disclosure, which include, but are not limited to, the following aspects.

One aspect is a method of modifying compliance with a treatment regimen by a subject having a chronic disease. The method includes obtaining a treatment regimen configured to manage the chronic disease, the treatment regimen including at least one action required to be performed by the subject using a subject computing device at one or more predetermined times; determining, using at least one computing devices, that the at least one action is performed by the subject at the one or more predetermined times; and if the at least one action is not performed by the subject at the one or more predetermined times, transmitting, using the at least one computing devices, a subject device control command to the subject computing device, the subject device control command configured to lock at least one function of the subject computing device for a predetermined release time and/or until the at least one action is performed by the subject. In embodiments, the subject is a child or adolescent and the locking of the function of the subject computing device for a period of time provides a disincentive to not comply with the treatment regimen, For example, if the subject is an adolescent and fails to take an action in accord with the treatment regimen, a mobile phone can be locked. Locking of at least one function on the mobile phone is a disincentive to the adolescent subject to not comply with the treatment regimen as it hinders social interaction with peers. A subject can unlock the function of the mobile phone by complying with the treatment regimen and taking the required action. In embodiments, if the subject complies with the treatment regimen, an incentive in the form of rewards or points can be accorded to the subject. In embodiments, a combination of incentives and disincentives can improve compliance with a treatment regimen. A treatment regimen can be also modified depending on the level of compliance.

Another aspect is a system for modifying compliance with a treatment regimen for a subject with a chronic disease. The system includes at least one processing device configured to control operation of the system, one or more computer readable data storage media storing software instructions that, when executed by the at least one processing device, cause the system to: obtain a subject treatment regimen configured to manage the chronic disease, the subject treatment regimen including at least one action required to be performed by the subject using a subject's computing device at one or more predetermined times; determine that the at least one action is performed by the subject at the one or more predetermined times; and if the at least one action is not performed by the subject at the one or more predetermined times, transmit a subject device control command to the subject computing device, the subject device control command configured to lock at least one function of the subject computing device for a predetermined release time and/or until the subject performs that at least one action.

Yet another aspect is a computer-readable storage medium comprising software instructions that, when executed, cause at least one processing device to: obtain a subject treatment regime configured to manage the chronic disease, the subject treatment regime including at least one action required to be performed by the subject using a subject computing device at one or more predetermined times; determine that the at least one action is performed by the subject at the one or more predetermined times; and if the at least one action is not performed by the subject at the one or more predetermined times, transmit a subject device control command to the subject computing device, the subject device control command configured to lock at least one function of the subject computing device for a predetermined release time and/or until the subject performs the at least one action.

Yet another aspect is a method of encouraging compliance with a treatment regimen by a subject with a chronic disease using a subject computing device. The method includes generating a notification of an action required by the treatment regimen using the subject computing device, the action required to be performed by the subject with a subject medical device at a predetermined time; if the action is not performed at the predetermined time, receiving a subject device control command from at least one computing device, the subject device control command configured to lock at least one function of the subject computing device; and disabling access to the at least one function of the subject computing device based on the subject device control command. In embodiments, the subject device control command can be initiated from computing device remote from the subject such as a server or laptop or can be initiated from the subject computing device. In other embodiments, a method includes generating a control command to accord the subject with incentives such as rewards or points upon performance of the action at the predetermined time.

The above features and advantages and other features and advantages of the present teachings are readily apparent from the following detailed description of the best modes for carrying out the present teachings when taken in connection with the accompanying drawings.

### BRIEF DESCRIPTION OF THE DRAWINGS

FIG. 1 illustrates an exemplary embodiment of a system for modifying compliance with a treatment regimen by a subject having a chronic condition.

FIG. 2 is a flowchart of an example method of operating the system of FIG. 1.

FIG. 3 is a flowchart illustrating an example method of performing an operation of FIG. 2.

FIG. 4 a block diagram that illustrates example motivational schemes.

FIG. 5 illustrates an exemplary architecture of a computing device that can be used to implement aspects of the present disclosure.

FIG. 6 is a flowchart of an example method of implementing the system of FIG. 1.

FIG. 7 is a block diagram of an example subject computing device, which communicates with an example subject medical device.

FIG. 8 is a block diagram illustrating example subject health data that are collected and generated by the subject computing device.

FIG. 9 is a flowchart illustrating an example method for operating the subject computing device.

FIG. 10 is a flowchart illustrating another example method for operating the subject computing device.

FIG. 11 is a block diagram that illustrates an example subject data management system.

FIG. 12 is a flowchart that illustrates an example method of operating a treatment regimen execution engine.

FIG. 13 is a flowchart illustrating an example method of operating a subject device control engine.

FIG. 14 is a flowchart illustrating another example method of operating the subject device control engine.

FIG. 15 is a flowchart that illustrates an example method of operating a health data analysis engine.

FIG. 16 is a flowchart that illustrates an example method of operating an incentive management engine.

### DETAILED DESCRIPTION

Various embodiments will be described in detail with reference to the drawings, wherein like reference numerals represent like parts and assemblies throughout the several views.

In general, the present disclosure is directed to systems and methods for modifying compliance with a treatment regimen by a subject having a chronic health condition. More specifically, the present disclosure is directed to systems and methods for encouraging a subject to comply with a treatment regimen to manage the subject's chronic disease, such as Type I diabetes, by utilizing incentive and/or disincentive devices. The disincentive device includes a mechanism for locking down a subject's computing device, such as a mobile device, if the subject has not complied with the treatment regimen. In some examples, the subject's computing device can be at least partially shut down (i.e., the subject is denied to access at least one function of the subject's computing device) and then automatically released after a predetermined amount of time. In other embodiments, the subject's computing device is released upon performing the action or after a predetermined amount of time. The incentive device includes a mechanism for providing rewards to the subject when the subject has complied with the treatment regimen.

For example, the systems and methods in accordance with the present disclosure are configured to monitor and incentivize a subject having Type I diabetics to routinely test their blood glucose levels as set forth in a treatment regimen. The subject uses a portable blood glucose meter to test a blood glucose level, and the blood glucose reading is transmitted to the diabetic's mobile device in real time. If it is determined that the testing has not been performed as planned in the treatment regimen, the subject's computing device, e.g. mobile device, can be locked down except for certain functions, such as the blood glucose testing functionality and emergency call to 911 or parents (or other interested parties). In some examples, the mobile device can be unlocked after a predetermined release time. In other examples, the mobile device can remain locked until a result of the scheduled testing is obtained by the mobile device, or until a predetermined action is performed by the subject, the parents, or other parties. In addition, the subject can earn rewards when they comply with the treatment regimen. Such rewards can be of various forms, such as reward points.

Accordingly, the systems and methods in accordance with the present disclosure modify and improve a subject's compliance with a treatment regimen. The incentive and disincentive devices of the present disclosure can be more effective than merely reminding patients about the treatment regimens because a subject can simply ignore or turn off the alarms or reminders. Further, the systems and methods allow the healthcare service provider to conveniently monitor and collect up-to-date information about the subject's medical and health conditions so that the healthcare service provider can make more proactive adjustment to the treatment regimen for the subject on a routine basis remotely. As a result, the subject need not visit the healthcare service provider as often.

This disclosure will be primarily described herein with exemplary embodiments of using blood glucose meters or blood-glucose measuring devices for use in assisting subjects with management of Type I diabetes. However, it should be understood by those skilled in the art that embodiments of the present disclosure extend to other medical tests or procedures. Therefore, although aspects of the present disclosure may be described with respect to blood glucose meters or measuring devices, the present disclosure should not be considered limited to those examples. For example, the incentive and disincentive devices can be employed as described herein with other chronic conditions for which lack of compliance with a treatment regimen is often observed such as asthma, cystic fibrosis, and sickle cell anemia.

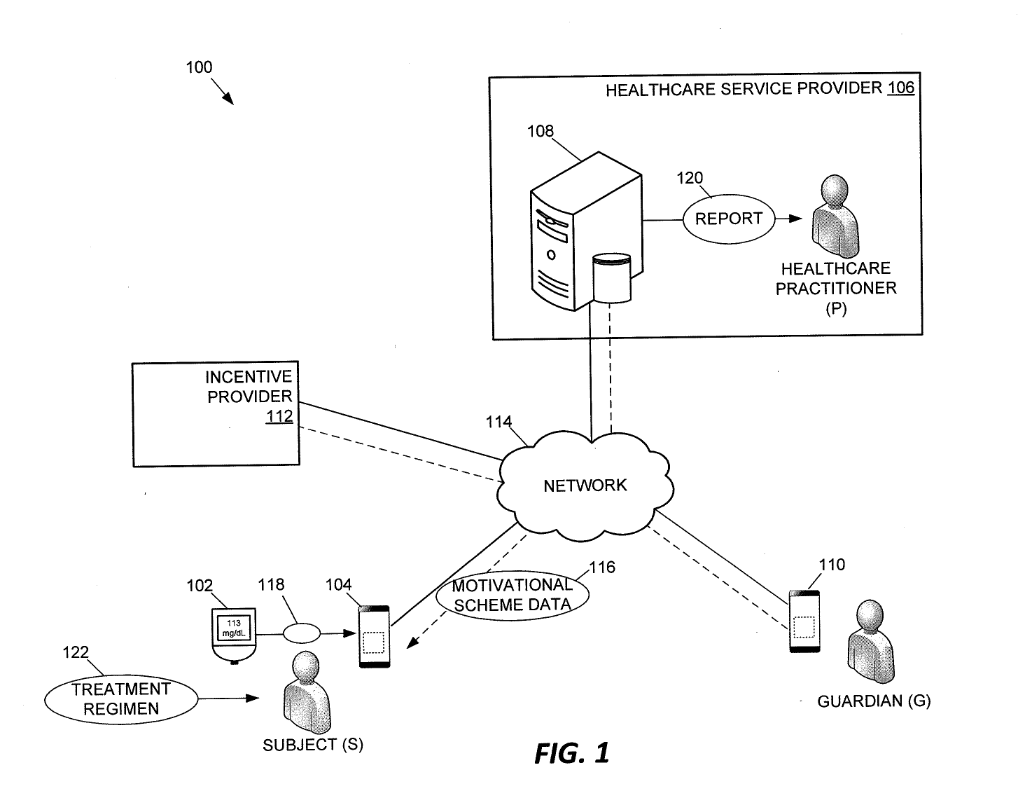

FIG. 1 illustrates an exemplary embodiment of a system 100 for encouraging compliance with a treatment regimen by a subject having a chronic condition. In this example, the system 100 includes a subject medical device 102 and a subject computing device 104, which are used by a subject S. The system 100 further includes a healthcare service provider 106 that manages a subject data management system 108. A guardian computing device 110 and an incentive provider 112 are also included in the system 100. Some embodiments of the system 100 can operate with a data communication network 114. Also shown are motivational scheme data 116.

In this disclosure, the system 100 is primarily described as a system for managing diabetes, such as Type I diabetes. However, as stated herein, the system 100 can be configured for other types of chronic diseases or health conditions, such as asthma cystic fibrosis, or sickle cell anemia treatment.

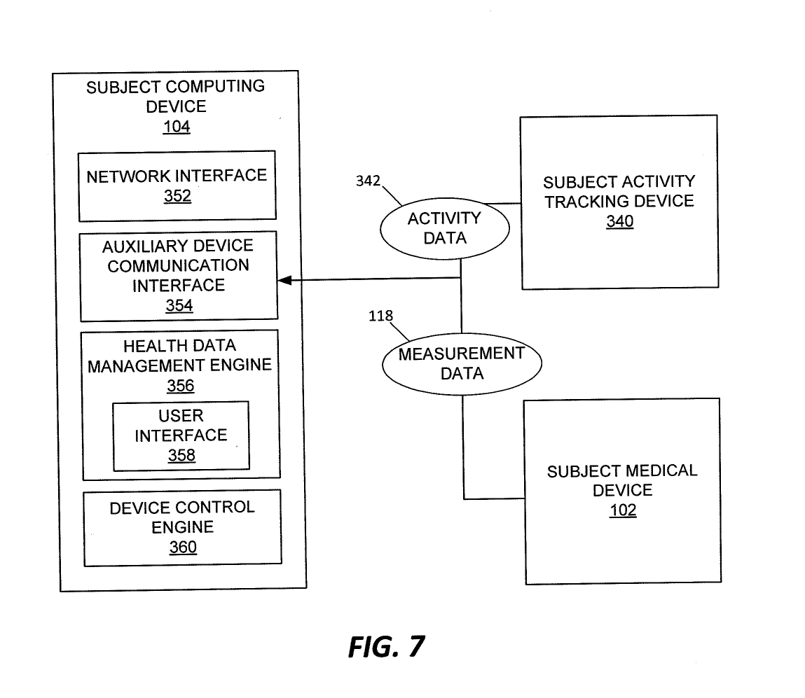

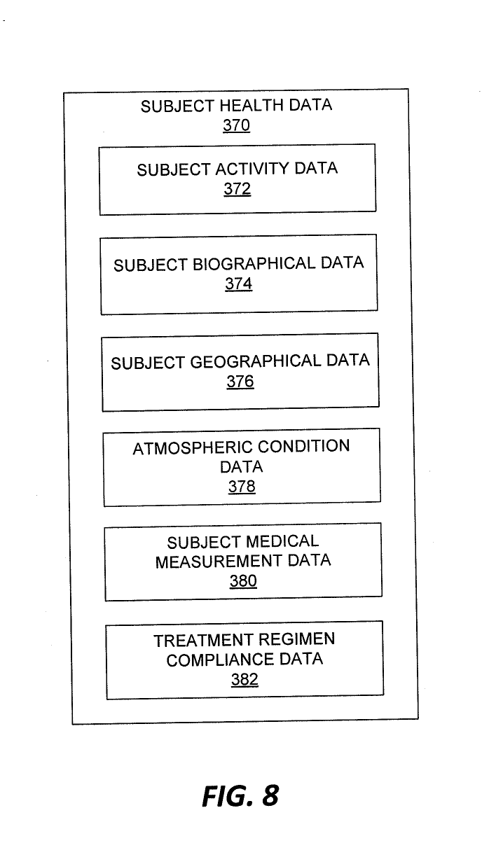

The subject medical device 102 is used by the subject S to measure the subject's health condition and generate measurement data 118. The measurement data 118 include information representative of the subject's health condition (e.g., blood glucose level). Some embodiments of the subject medical device 102 include a display screen configured to display the measurement data 118 thereon. As shown in FIG. 7 in more detail, the subject medical device 102 can be connected to the subject computing device 104 to transmit the measurement data to the subject computing device 104. The subject computing device 104 can then communicate with the subject data management system 108 of the healthcare service provider 106 over the network 114. For example, the subject computing device 104 can transmit the measurement data 118 (and other data, such as activity data 342 as shown in FIG. 7 or health data 370 as shown in FIG. 8) to the subject data management system 108 through the network 114. In some embodiments, the subject computing device 104 can independently analyze the measurement data 118 when the measurement data 118 is transmitted from the subject medical device 102.

The subject medical device 102 can be of various types, depending on the subject's chronic diseases to be cared for. Where diabetes is concerned, the subject medical device 102 can be a blood glucose meter or other device for measuring blood glucose. A blood glucose meter is a medical device for determining the concentration of glucose in the blood. Some embodiments of blood glucose meters are portable for personal use. Blood glucose meters are of various types. One example glucose meter uses disposable test strips on which a small drop of blood is placed, and reads the strips to calculate the blood glucose level. In some embodiments, the glucose meter includes a display screen configured to display glucose readings thereon. Other types and/or configurations of glucose meters are possible in other embodiments.

Other types of the subject medical device 102 can also be used for other purposes. For example, the subject medical device 102 is an insulin injection device (e.g., an insulin pen) for diabetes; an artificial pancreas device for diabetes; a vest machine for patients with cystic fibrosis; a nebulizer or an inhaler for patients with cystic fibrosis, asthma, COPD, and other respiratory diseases, or a device that measure hemoglobin.

Although it is primarily described in this disclosure that the subject medical device 102 is a blood glucose meter, it should be understood by those skilled in the art that the subject medical device 102 can be of other types for monitoring and/or treating other health conditions or diseases.

The subject computing device 104 is used by the subject S and configured to receive the measurement data 118 (i.e., the information regarding the subject's health condition, such as a blood glucose level) from the subject medical device 102 and/or the measurement data 118 is entered by the subject S into the subject computing device and stored and analyzed using other application programs. The subject computing device 104 is also configured to communicate with the subject data management system 108. In some embodiments, the subject computing device 104 operates to transmit the measurement data 118 and other data associated with the subject S to the subject data management system 108, and receive the motivational scheme data 116 from the subject data management system 108. An example of the subject computing device 104 is described and illustrated in more detail with reference to FIG. 7. In embodiments, a subject computing device includes a mobile phone, a tablet computer, an internet enabled television, an internet enabled gaming system, and a computer. Upon compliance with the treatment regimen, e.g. glucose blood testing, the subject computing device sends the data to the health care service provider. Upon receipt of the data in accord with the predetermined regimen and at a predetermined time a reward or points are accorded to the subject.

The healthcare service provider 106 is a person, entity, institution, or agency that provides healthcare service to the subject S. Examples of healthcare service provider 106 include primary care providers (e.g., doctors, nurse practitioners, and physician assistants), nursing care providers (e.g., nurses), specialty care providers (e.g., professionals in various specialties), and health professionals or institutions that provide preventive, curative, promotional and rehabilitative health care services.

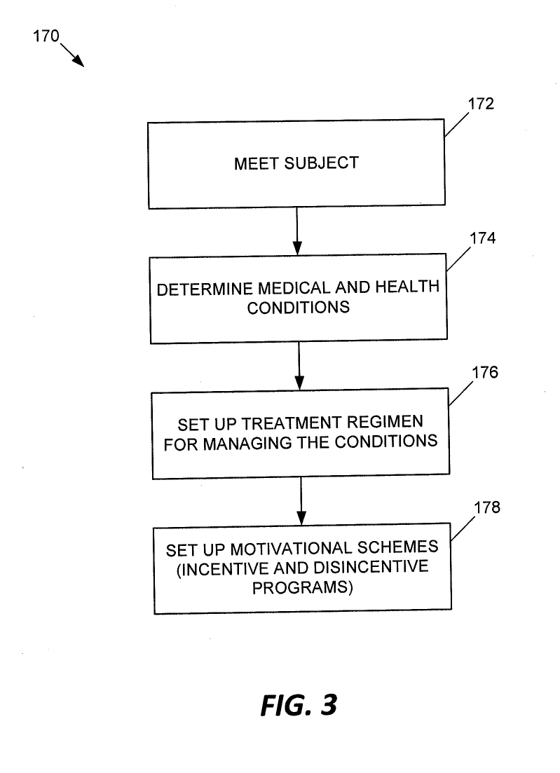

For example, the healthcare service provider 106 can provide various services for the subject S having diabetes. The healthcare service provider 106 can include a doctor who diagnoses the diabetes. The healthcare service provider 106 can set up a treatment plan (e.g., a treatment regimen 122) for monitoring and treating the diabetes. As described herein, some embodiments of a treatment plan includes schedules for blood glucose monitoring, dietary plans, exercise plan, and medications. In embodiments, the treatment regimen includes glucose testing at a predetermined times, taking medications including insulin at predetermined times or in response to blood sugar levels, and eating at predetermined schedules or in response to blood glucose levels. An example method of setting up a treatment regimen is described and illustrated in more detail with reference to FIG. 3.

The subject data management system 108 operates to monitor the subject's health conditions and other information (e.g., subject health data 370 (FIG. 8)) and provide motivational scheme data 116 for the subject S. The subject data management system 108 can be operated by the healthcare service provider 106. For example, the subject data management system 108 can be managed by the healthcare service provider 106 either directly or through a third party operator.

Some embodiments of the subject data management system 108 are configured to receive the measurement data 118 (and other data associated with the subject S) from the subject computing device 104, and analyze the data for various purposes. In some embodiments, the subject data management system 108 operates to generate a subject health data report 120 based on the analysis. As described herein, the subject health data report 120 is configured to assist a healthcare practitioner P to provider a healthcare service to the subject S. Some embodiments of the subject health data report 120 include information customized for the subject S. In some embodiments, other information is obtained from other sources or devices. In some embodiments, other sources include other data analysis applications present in the subject computing device, e.g. diabetes management tools. In some embodiments, another device that measures health status of the subject includes a fitness tracker, a mobile phone, and an external blood pressure monitor. For example, the subject health data report 120 can include information about health conditions, exercise, diet, food and beverage intake, food and beverage contents (e.g. sugar content), dosage of drugs administered, diagnosis of diseases, and/or recommendations or prescriptions for the subject S.

In some embodiments, the subject health data report 120 can be delivered to one or more healthcare practitioners P. The healthcare practitioners P can refer to the subject health data report 120 to determine the conditions of the subject S and care for the subject S based on the conditions. For example, the healthcare practitioners P can be physicians, nurses, health educators, community health workers, and other healthcare practitioners or providers.

The guardian computing device 110 is used by a guardian G and operates to receive data from the subject data management system 108. In some embodiments, such data include information representing whether the subject S has performed an action required by the treatment regimen 122. In other embodiments, the data include information about the subject's health conditions (including the measurement data 118), so that the guardian G is updated with the status or conditions of the subject S.

In some embodiments, the guardian G is a person or a group of people who are interested in the health conditions of the subject S. Examples of the guardian G include a parent or guardian of the subject S, a family member of the subject S, a primary physician of the subject S, a health care manager, and any other interested parties.

The incentive provider 112 is a person or entity that runs one or more incentive programs for the subject S. As part of an incentive program for motivating the subject S to comply with the treatment regimen 122, the incentive provider 112 can cooperate with the healthcare service provider 106 and provide incentives or rewards the subject's compliance with the treatment regimen 122. The incentive provider 112 can be of various types. For example, where the healthcare service provider 106 provides rewards or points to the subject S as the subject S meets the requirements of the treatment regimen 122, the incentive provider 112 can be one or more businesses (e.g., online/offline retailers, online game stores, philanthropic organizations, and insurance companies) that provide goods or services to the subject S in exchange for the rewards or points. In some embodiments, the healthcare service provider 106 is partnered with the incentive provider 112 to provide incentives to the subject S. In other embodiments, the healthcare service provider 106 can operate as the incentive provider 112, without cooperating with one or more incentive providers 112 outside the healthcare service provider 106. In embodiments, the incentive provider and/or health care provider provides a real time report of rewards or points to the subject's computing device. In embodiments, the incentive provider is a parent, guardian, or family member. In embodiments, a subject is a child or adolescent, and incentives are designed to increase compliance with the treatment system.

The data communication network 114 communicates digital data between one or more computing devices, such as among the subject computing device 104, the subject data management system 108, the guardian computing device 110, and/or the incentive provider 112. Examples of the network 114 include a local area network and a wide area network, such as the Internet.

In some embodiments, the network 114 includes a wireless communication system, a wired communication system, or a combination of wireless and wired communication systems. A wired communication system can transmit data using electrical or optical signals in various possible embodiments. Wireless communication systems typically transmit signals via electromagnetic waves, such as in the form of optical signals or radio frequency (RF) signals. A wireless communication system typically includes an optical or RF transmitter for transmitting optical or RF signals, and an optical or RF receiver for receiving optical or RF signals. Examples of wireless communication systems include Wi-Fi communication devices (such as utilizing wireless routers or wireless access points), cellular communication devices (such as utilizing one or more cellular base stations), and other wireless communication devices.

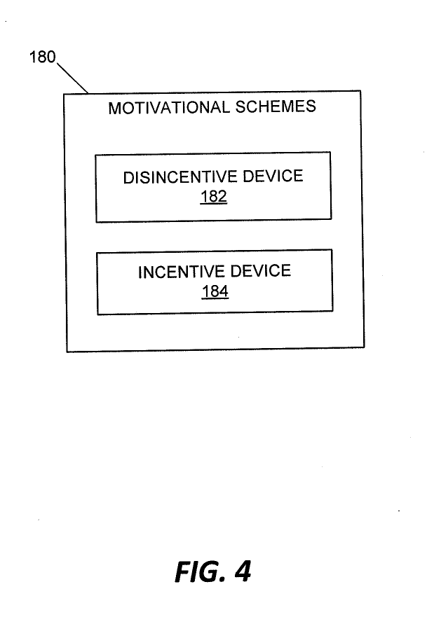

The motivational scheme data 116 are usable in the system 100 to encourage or assist the subject S to comply with the treatment regimen designed to manage the subject's chronic disease. An example of the motivational scheme data 116 is described and illustrated in more detail with reference to FIG. 4.

The measurement data 118 include information about the subject's health condition that is measured by the subject medical device 102. The subject's health condition can be of various types and represented in various manners. Where the subject medical device 102 is a blood glucose meter, the measurement data 118 can include a blood glucose level of the subject S that is measured by the blood glucose meter. Other information can be included in the measurement data 118 depending on the types of the subject medical device 102 and/or other data input entered by the subject.

The treatment regimen 122 is configured to manage and/or treat the subject's chronic disease. In some embodiments, the treatment regimen 122 includes information about a course of medical treatment, health plans, and/or dietary plans, which are customized for the subject S.

Some embodiments of the treatment regimen 122 include a schedule for at least one action to be performed by the subject S. In particular, the treatment regimen 122 can include at least one action that is to be performed using the subject medical device 102 and/or the subject computing device 104 at one or more predetermined times. For example, the treatment regimen 122 requires the subject S to, at one or more predetermined times or intervals, measure his or her blood glucose level using the subject medical device 102 (e.g., a blood glucose meter) and transmit the measurement (e.g., the measurement data 118) to the subject data management system 108 using the subject computing device 104.

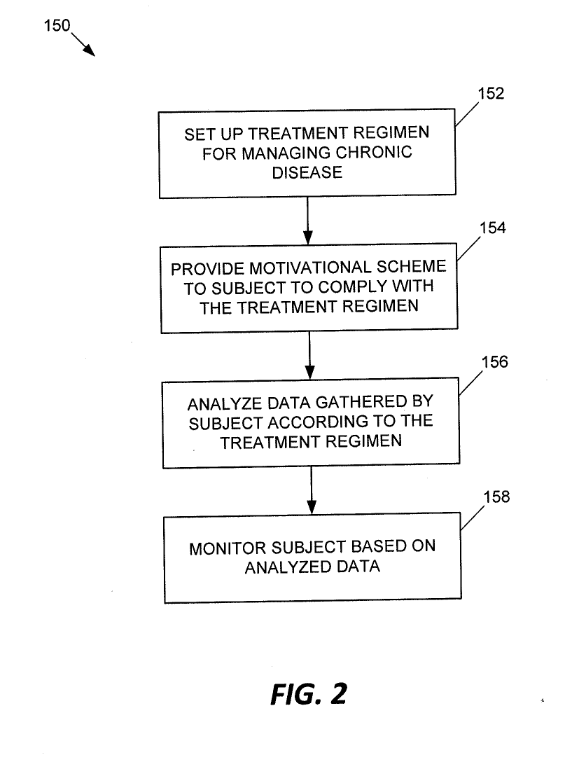

FIG. 2 is a flowchart of an example method 150 of operating the system 100.

The method 150 can begin with operation 152, in which the healthcare service provider 106 sets up the treatment regimen 122 for managing the subject's chronic disease. An example method of performing the operation 152 is further described with reference to FIG. 3.

At operation 154, the healthcare service provider 106 (e.g., the subject data management system 108) provides motivational schemes (e.g., in the form of the motivational scheme data 116) to the subject S (e.g., through the subject computing device 104). The motivational schemes are designed to encourage and assist the subject S to comply with the treatment regimen 122. An example of the motivational schemes is described in more detail with reference to FIG. 4.

At operation 156, the subject data management system 108 operates to analyze data gathered by the subject S in accordance with the treatment regimen 122. As described herein, the treatment regimen 122 can include a schedule for the subject S to take at least one action to comply with the treatment regimen 122. For example, such an action can be measuring the subject's blood glucose level at a prescribed schedule. The subject S is encouraged and assisted by the motivational schemes to take such an action as planned in the treatment regimen 122.

In some embodiments, the subject data management system 108 operates to determine trends shown in the measurements and generate a recommendation for the subject S to manage the subject's chronic disease and/or modify the motivational schemes.

At operation 158, the healthcare service provider 106 can monitor the subject S based on the analyzed data (e.g., information associated with the subject's blood glucose level). As described herein, the healthcare practitioner P can use the analyzed data that are provided to the healthcare practitioner P in the form of, for example, the subject health data report 120, and provide appropriate care to the subject S. In other examples, the guardian G can be provided with the analyzed data so that the guardian G refers to the analyzed data to monitor and assist the subject S in complying with the treatment regimen 122.

FIG. 3 is a flowchart illustrating an example method 170 of performing the operation 152 of FIG. 2. In some embodiments, the method 170 begins with operation 172, in which the healthcare service provider 106 meets the subject S. For example, the subject S having a chronic disease or other medical or health conditions can visit the healthcare service provider 106 to receive diagnosis and treatment from the healthcare service provider 106.

At operation 174, the healthcare service provider 106 determines medical and/or health conditions of the subject S through various procedures, such as health history and screening questionnaire and medical tests. For example, diabetes can be diagnosed by a blood test, such as an HbA1C test, fasting plasma glucose (FPG) test, or an oral glucose tolerance test (OGTT).

At operation 176, the healthcare service provider 106 can set up a treatment regimen 122 for managing the subject conditions, such as a chronic disease. The healthcare service provider 106 can confer with the subject S and/or the guardian G to better customize the treatment regimen 122 for the particular subject S. As described herein, some embodiments of a treatment plan includes schedules for blood glucose monitoring, dietary plans, exercise plan, and medications. In embodiments, the treatment regimen includes glucose testing at a predetermined times, taking medications including insulin at predetermined times or in response to blood sugar levels, and eating at predetermined schedules or in response to blood glucose levels.

At operation 178, the healthcare service provider 106 can set up motivational schemes for encouraging and assisting the subject S to comply with the treatment regimen 122. As described in more detail with reference to FIG. 4, the motivational schemes include various incentive and disincentive programs. In some embodiments, such motivational schemes can be implemented using at least one of the subject medical device 102, the subject computing device 104, the healthcare service provider 106 including the subject data management system 108, the guardian computing device 110, the incentive provider 112, and any other suitable computing devices. In embodiments, a motivational scheme includes reminders and/or warnings sent to the subject's computing device to take an action of the treatment regimen, a recognition that the action was taken or not taken, rewards or points accorded to subject if the action was taken in accord with the treatment regimen, and a lockdown command if the action is not taken within a predetermined time. In embodiments, the subject is a child or an adolescent and the incentive and disincentive programs are designed to increase compliance of such subjects with the treatment regimen.

FIG. 4 a block diagram that illustrates example motivational schemes 180. In some embodiments, the motivational schemes 180 include a disincentive device 182 and an incentive device 184.

The motivational schemes 180 are designed to modify the subject's compliance with the treatment regimen 122. For example, the motivational schemes 180 are used to encourage the subject S to routinely test his or her blood glucose levels as prescribed in the treatment regimen 122.

The disincentive device 182 is configured to provide disincentives to the subject S when the subject S does not comply with the treatment regimen 122. In some embodiments, such disincentives can be implemented using at least one of the subject medical device 102, the subject computing device 104, the healthcare service provider 106 including the subject data management system 108, the guardian computing device 110, the incentive provider 112, and any other suitable computing devices. As described herein, some embodiments of the disincentive device 182 are configured to lock down at least some features of the subject computing device 104, such as the subject's mobile device, when the subject S does not take an action as required by the treatment regimen 122. In embodiments, when the subject fails to take action at a predetermined time, a lock down command is sent to the subject's computing device. As the subject computing device 104 (e.g., mobile devices) is typically seen as an essential device for a personal and/or social life, restricting the subject from accessing the subject computing device 104 can provide a significant disincentive to overlooking or ignoring the treatment regimen 122. In embodiments, when a subject is a child or adolescent, lock down of the subject computing device can increase compliance with the treatment regimen.

In some embodiments, the disincentive device 182 includes a device control command 186. Once transmitted to the subject computing device 104, the device control command 186 is configured to enable the subject computing device 104 to lock at least one function of the subject computing device 104. In some embodiments, the device control command 186 is configured to lock at least one function of the subject computing device 104 for a predetermined release time or until a predetermined action is performed by the subject S. In embodiments, a predetermined release time is at least 1 hour to 24 hours or more. Some embodiments of the device control command 186 are configured to disable access to all functions of the subject computing device 104 except for essential functions.

The subject computing device 104 can be configured to perform various functions. For example, where the subject computing device 104 is configured as a smartphone or tablet, the functions include a mobile telephone, various mobile applications with a variety of services or features, device native functions (e.g., camera), device native applications, access to a system account, and access to service accounts (e.g., Facebook, Twitter, Google accounts, App Store accounts, etc.). Where the subject computing device 104 is configured as a gaming system or entertainment system, the functions include access to the subject's personal account associated with gaming or entertainment items (e.g., games, music files, picture files, or movie files), log-in features, and other functions running on gaming systems or entertainment systems.

The essential functions of the subject computing device 104 include functions that assist the subject to take an action as planned, or unlock the subject computing device 104. Where the subject computing device 104 is configured as a smartphone or other devices having voice or video call functions, examples of such essential functions include at least one of an emergency call option, a call to the healthcare service provider 106 (e.g., the healthcare practitioner P), a call to the guardian G, and a function that allows the subject to take an action as required by the treatment regimen (e.g., taking blood glucose measurement using a blood glucose meter). Where the subject computing device 104 is configured as a gaming system or entertainment system, the essential functions of the subject computing device 104 can include text or voice message functionality to the healthcare service provider 106 and/or the guardian G, and/or a function that allows the subject to take an action as required by the treatment regimen.

The incentive device 184 is configured to provide incentives to the subject S when the subject S meets the requirements in the treatment regimen 122. As described herein, some embodiments of the incentive device 184 are configured to provide rewards to the subject S when the subject S complies with the required testing and/or other actions as required in the treatment regimen 122. Rewards or points can be increased upon increasing compliance. When the health care provider receives data that the action was taken, rewards or point are accorded to the subject. The health care provider and/or incentive provider can report the status or amount of reward points to the subject's computing devices.

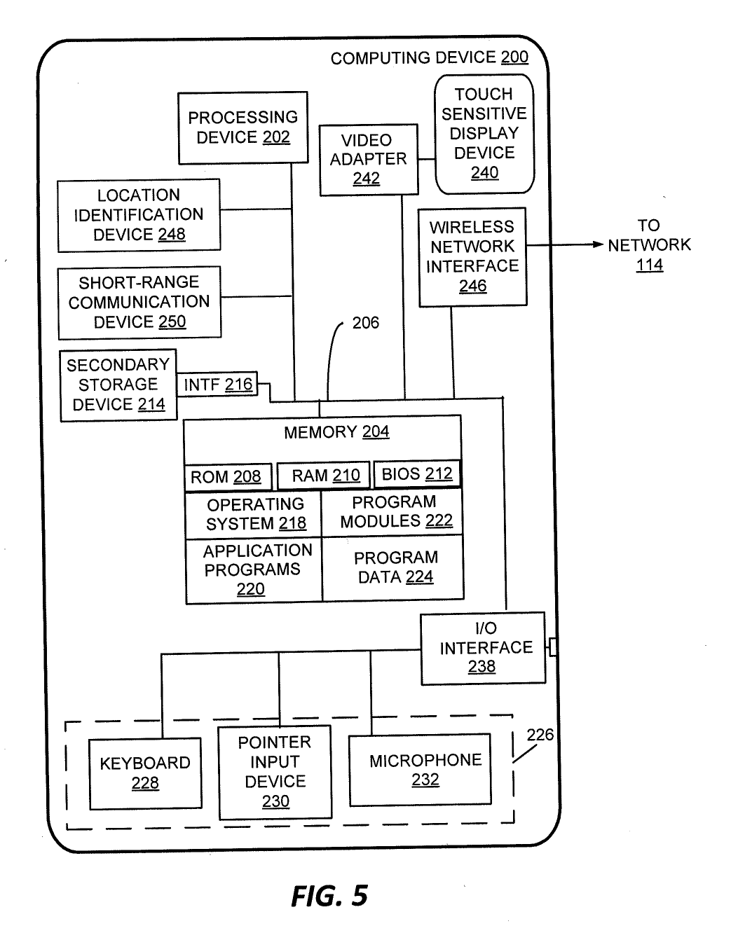

FIG. 5 illustrates an exemplary architecture of a computing device that can be used to implement aspects of the present disclosure, including the subject medical device 102, the subject computing device 104, the subject data management system 108, and the guardian computing device 110, and will be referred to herein as the computing device 200. The computing device 200 is used to execute the operating system, application programs, and software modules (including the software engines) described herein.

The computing device 200 includes, in some embodiments, at least one processing device 202, such as a central processing unit (CPU). A variety of processing devices are available from a variety of manufacturers, for example, Intel or Advanced Micro Devices. In this example, the computing device 200 also includes a system memory 204, and a system bus 206 that couples various system components including the system memory 204 to the processing device 202. The system bus 206 is one of any number of types of bus structures including a memory bus, or memory controller; a peripheral bus; and a local bus using any of a variety of bus architectures.

Examples of computing devices suitable for the computing device 200 include a desktop computer, a laptop computer, a tablet computer, a mobile device (such as a smart phone, an iPod® mobile digital device, or other mobile devices), internet enabled television, internet enabled gaming system, or other devices configured to process digital instructions.

The system memory 204 includes read only memory 208 and random access memory 210. A basic input/output system 212 containing the basic routines that act to transfer information within computing device 200, such as during start up, is typically stored in the read only memory 208.

The computing device 200 also includes a secondary storage device 214 in some embodiments, such as a hard disk drive, for storing digital data. The secondary storage device 214 is connected to the system bus 206 by a secondary storage interface 216. The secondary storage devices and their associated computer readable media provide nonvolatile storage of computer readable instructions (including application programs and program modules), data structures, and other data for the computing device 200.

Although the exemplary environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media.

A number of program modules can be stored in secondary storage device 214 or memory 204, including an operating system 218, one or more application programs 220, other program modules 222, and program data 224.

In some embodiments, computing device 200 includes input devices to enable a user to provide inputs to the computing device 200. Examples of input devices 226 include a keyboard 228, pointer input device 230, microphone 232, and touch sensitive display 240. Other embodiments include other input devices 226. The input devices are often connected to the processing device 202 through an input/output interface 238 that is coupled to the system bus 206. These input devices 226 can be connected by any number of input/output interfaces, such as a parallel port, serial port, game port, or a universal serial bus. Wireless communication between input devices and interface 238 is possible as well, and includes infrared, BLUETOOTH® wireless technology, 802.11a/b/g/n, cellular, or other radio frequency communication systems in some possible embodiments.

In this example embodiment, a touch sensitive display device 240 is also connected to the system bus 206 via an interface, such as a video adapter 242. The touch sensitive display device 240 includes touch sensors for receiving input from a user when the user touches the display. Such sensors can be capacitive sensors, pressure sensors, or other touch sensors. The sensors not only detect contact with the display, but also the location of the contact and movement of the contact over time. For example, a user can move a finger or stylus across the screen to provide written inputs. The written inputs are evaluated and, in some embodiments, converted into text inputs.

In addition to the display device 240, the computing device 200 can include various other peripheral devices (not shown), such as speakers or a printer.

When used in a local area networking environment or a wide area networking environment (such as the Internet), the computing device 200 is typically connected to the network through a network interface, such as a wireless network interface 246. Other possible embodiments use other communication devices. For example, some embodiments of the computing device 200 include an Ethernet network interface, or a modem for communicating across the network.

The computing device 200 typically includes at least some form of computer-readable media. Computer readable media includes any available media that can be accessed by the computing device 200. By way of example, computer-readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 200.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

Referring again to FIG. 5, the computing device 200 can include a location identification device 248. The location identification device 248 is configured to identify the location or geolocation of the computing device 200. The location identification device 248 can use various types of geolocating or positioning systems, such as network-based systems, handset-based systems, SIM-based systems, Wi-Fi positioning systems, and hybrid positioning systems. Network-based systems utilize service provider's network infrastructure, such as cell tower triangulation. Handset-based systems typically use the Global Positioning System (GPS). Wi-Fi positioning systems can be used when GPS is inadequate due to various causes including multipath and signal blockage indoors. Hybrid positioning systems use a combination of network-based and handset-based technologies for location determination, such as Assisted GPS.

Referring again to FIG. 5, the computing device 200 further includes a short-range wireless communication device 250. The short-range wireless communication device 250 is configured to establish short-range wireless communication with adjacent computing devices. For example, the short range wireless communication device 250 can be used to establish short-range wireless communication between the subject medical device 102 and the subject computing device 104. Short-range wireless communication is one-way or two-way short-range to medium-range wireless communication. Short-range wireless communication can be established according to various technologies and protocols. Examples of short-range wireless communication include a radio frequency identification (RFID), a near field communication (NFC), a Bluetooth technology, and a Wi-Fi technology.

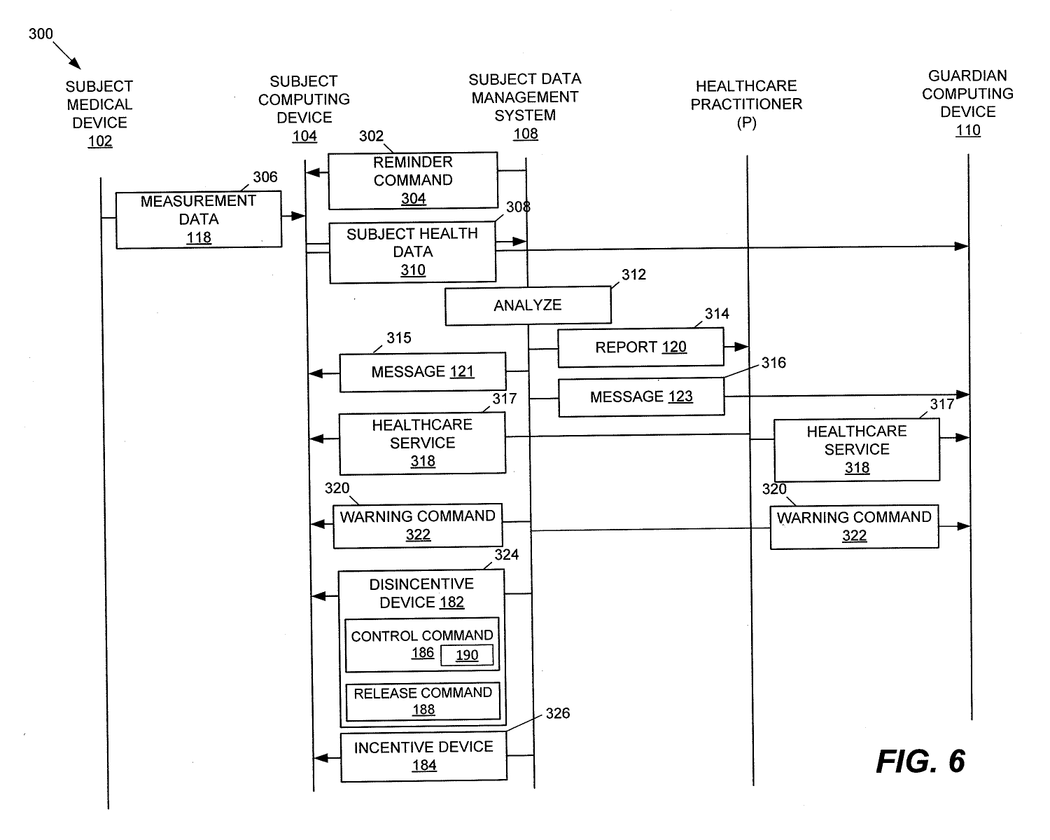

FIG. 6 is a flowchart of an example method 300 of implementing the system 100. The method 300 is described with an example relation with the subject medical device 102, the subject computing device 104, the subject data management system 108, the healthcare practitioner P, and the guardian computing device 110.

In this example, the method 300 starts with operation 302, in which the subject data management system 108 transmits a reminder command 304 to the subject computing device 104 before a predetermined time at which the subject S is required to take an action as scheduled by the treatment regimen 122. The reminder command 304 is used to enable the subject computing device 104 to generate at least one notification of the scheduled action and present the notification on the subject computing device 104 so that the subject S recognize the notification ahead of the predetermined time. For example, the reminder command 304 is configured to present the notification a preset time before the predetermined time. Such a preset time can vary depending on different situations, and can be customized by the healthcare service provider 106, the subject S, and/or the guardian G. Examples of the preset time are one to ten to ten minutes, and other times are also possible for the preset time.

Once the subject S receives the reminder notification through the subject computing device 104 and perform the action (e.g., testing a blood glucose level) as scheduled using the subject medical device 102, the subject medical device 102 transmits the measurement data 118 to the subject computing device 104 at operation 306. As described herein, the measurement data 118 include information about the subject's health condition that is measured using the subject medical device 102.

At operation 308, the subject computing device 104 receives the measurement data 118 and generates subject health data 310 based on the measurement data 118 and other data associated with the subject S. The subject computing device 104 can transmit the subject health data 310 to the subject data management system 108. In some embodiments, the subject computing device 104 can also transmit the subject health data 310 to the guardian computing device 110 for the guardian's reference. An example of the subject health data 310 is described in more detail with reference to FIG. 8.

At operation 312, the subject data management system 108 operates to analyze the subject health data 310 to monitor the status or condition of the subject's chronic disease. In some embodiments, the subject data management system 108 can evaluate trends found in the subject health data 310 and determine any abnormality found in the trends. For example, the subject data management system 108 monitors a blood glucose level of the subject S and see if the level changes beyond a predetermined safe range.

At operation 314, the subject data management system 108 generates the subject health data report 120 based on the analysis of the operation 312, and transmit the report 120 to the healthcare practitioner P. The healthcare practitioner P can refer to the subject health data report 120 when providing healthcare service to the subject S and/or modify the treatment regimen, incentives, and/or disincentives.

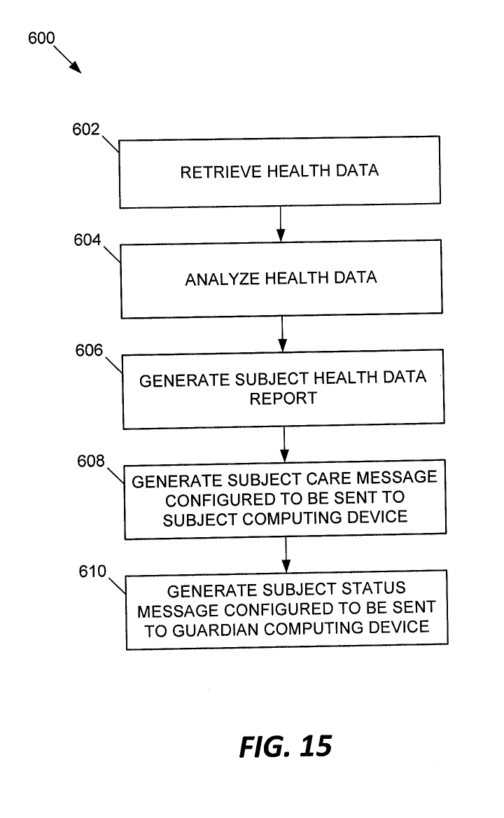

At operation 315, the subject data management system 108 transmits a subject care message 121 to the subject computing device 104. The subject care message 121 can be generated based on the analysis at the operation 312 and include information about the subject's status and conditions, and recommendations to improve them. An example of the subject care message 121 is described in more detail with reference to FIG. 15.

At operation 316, the subject data management system 108 transmits a subject status message 123 to the guardian computing device 110. The subject status message 123 can be generated based on the analysis at the operation 312 and include information about the subject's status and conditions, and recommendations to improve them. An example of the subject status message 123 is described in more detail with reference to FIG. 15.

At operation 317, the healthcare practitioner P can provide healthcare service 318 to the subject S using the subject computing device 104 and/or the guardian G using the guardian computing device 110. The subject health data report 120 can be used to determine the healthcare service 318 that is appropriate for the subject S. Examples of the healthcare service 318 include seeing the subject S and the guardian G, prescribing medication, proposing treatment regimens personalized for the subject S, monitoring the subject's conditions and status, modifying the treatment regimen, modifying the compliance scheme to increase incentive or disincentives, and other suitable services for taking care of the subject S. For example, if the subject fails to test their blood sugar at the predetermined time for two to three times in a row, the lockdown time of the mobile device can be increased for 1 hour to 24 hours. If the subject increase the number of times testing is done in accord with the treatment regimen, the value of the reward points may be increased.

In some embodiments, the method 300 can include operation 320, in which the subject data management system 108 transmits a warning command 322 to the subject computing device 104 shortly before the predetermined time at which the subject S is required to take an action as scheduled by the treatment regimen 122. Similarly to the reminder command 304, the warning command 322 is configured to enable the subject computing device 104 to generate and present at least one warning notification on the subject computing device 104 to alarm that the scheduled time is approaching and the subject S needs to take the action very shortly. In some embodiments, the subject data management system 108 can transmit the warning command 322 to the guardian computing device 110 at the same time, or at a similar time, when it sends the warning command 322 to the subject computing device 104. In this case, the warning command 322 also enables the guardian computing device 110 to generate and present at least one warning notification on the guardian computing device 110 to inform that the scheduled time is approaching and the subject S needs to take the action very shortly.

In other embodiments, the operation 320 is performed such that the subject data management system 108 transmits a warning command 322 to the subject computing device 104 and/or the guardian computing device 110 after the predetermined time at which the subject S is required to take an action as scheduled by the treatment regimen 122. The warning command 322 is configured to enable the subject computing device 104 and/or the guardian computing device 110 to generate and present at least one warning notification on the subject computing device 104 and/or the guardian computing device 110 to inform that the scheduled action has not been performed by the subject S at the predetermined time.

At operation 324, the subject data management system 108 operates to provide the disincentive device 182 through the subject computing device 104 so as to disincentivize the subject S when the subject S does not comply with the treatment regimen 122. An example of the disincentive device 182 is illustrated and described herein.

At operation 326, the subject data management system 108 operates to provide the incentive device 184 through the subject computing device 104 so as to incentivize the subject S when the subject S complies with the treatment regimen 122. An example of the incentive device 184 is illustrated and described herein.

FIG. 7 is a block diagram of an example subject computing device 104, which communicates with the subject medical device 102. In this example, a subject activity tracking device 340 is further provided to transmit activity data 342 to the subject computing device 104.

In some embodiments, the system 100 can further employ the subject activity tracking device 340, as well as the subject computing device 104, in order to gather various pieces of information associated with the subject S. For example, the subject activity tracking device 340 is used by the subject S and configured to monitor and track fitness and/or health related metrics, such as activity amount (e.g., exercise time, steps, and/or distance walked or run), calorie consumption, heartbeat, and quality of sleep. The subject activity tracking device 340 is configured to be connected and synchronized, either wired or wirelessly, to the subject computing device 104. In some embodiments, the subject activity tracking device 340 is configured to be wearable by the subject S as, for example, a wrist band, wrist watch, or clip-on.

The activity data 342 are generated by the subject activity tracking device 340 and include information associated with the subject's activities, such as activity time, steps, distance walked or run, calorie consumption, heartbeat, heart rate, quality of sleep (e.g., tracks length, sleep amount, sleep deepness, and length of REM sleep), skin perspiration, skin temperature, and blood oxygenation level (SpO2).

In this example, the subject computing device 104 includes a network interface 352, an auxiliary device communication interface 354, a health data management engine 356 including a user interface 358, and a device control engine 360.

The network interface 352 is an interface that operates to communicate with other computing devices through the network 114. In some embodiments, the network interface 352 is configured similarly to the wireless network interface 246 as described in FIG. 5.

The auxiliary device communication interface 354 is an interface that operates to communicate with other computing devices near the subject computing device 104, such as the subject medical device 102 and the subject activity tracking device 340. In some embodiments, the auxiliary device communication interface 354 is configured similarly to the I/O interface 238, and/or the short-range communication device 250, as shown in FIG. 5. The auxiliary device communication interface 354 can operate to synchronize the subject medical device 102 and the subject activity tracking device 340 to the subject computing device 104. In some embodiments, the auxiliary device communication interface 354 is configured to enable the subject computing device 104 to synchronize with the subject medical device 102 and/or the subject activity tracking device 340 in real time. As soon as the measurement data 118 and the activity data 342 are created or updated in the subject medical device 102 and the subject activity tracking device 340, respectively, the auxiliary device communication interface 354 can operate to obtain the data 118 and 342 instantaneously. In other embodiments, the auxiliary device communication interface 354 is configured to periodically access the subject medical device 102 and/or the subject activity tracking device 340 and determine whether the measurement data 118 and the activity data 342 have been newly created or updated, and receive the data 118 and 342. In other embodiments, measurement data can be input into an application program in the subject computing device and stored until the measurement data is sent to the subject data management system.

The health data management engine 356 operates to receive the measurement data 118 from the subject medical device 102, and/or the activity data 342 from the subject activity tracking device 340, and analyze the measurement data 118 and/or the activity data 342. Where the subject medical device 102 is a blood glucose meter, the health data management engine 356 can analyze the subject's blood glucose level and other information, such as carbohydrates level, food types, insulin level, and medication intake. The health data management engine 356 can further operate to track and analyze a trend or progress of one or more particular readings, such as a blood glucose level or insulin level, of the subject S.

In some embodiments, the health data management engine 356 generates a subject health data 370 (FIG. 8) based on the measurement data 118, the activity data 342, and/or other data obtained by the subject computing device 104 or other devices. The subject health data 370 can then be transmitted from the subject computing device 104 to the subject data management system 108 for further analysis. An example of the subject health data 370 is described with reference to FIG. 8.

The user interface 358 provides an interface for a user, such as the subject S, to interact with the subject computing device 104, in particular the health data management engine 356. For example, the user interface 358 provides a graphic user interface displayed on a touch screen of the subject computing device 104 so that the subject S can see the information displayed thereon, navigate various menu or options provided by the health data management engine 356, and enter various inputs to the subject computing device 104.

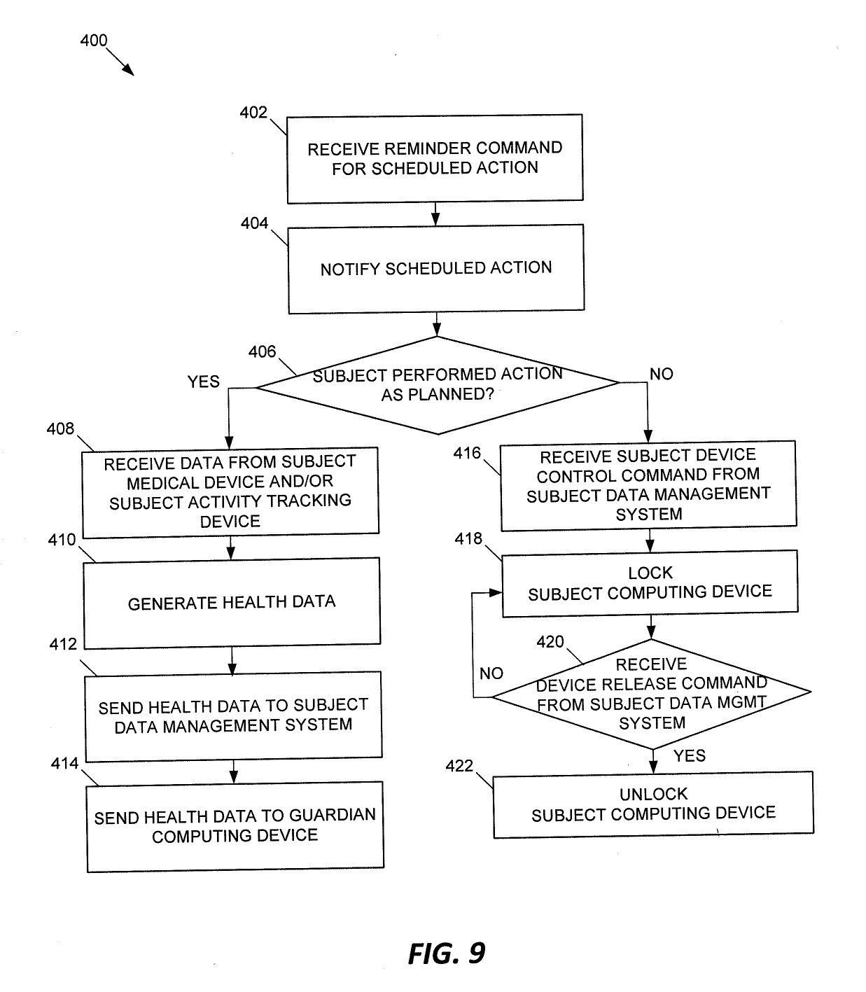

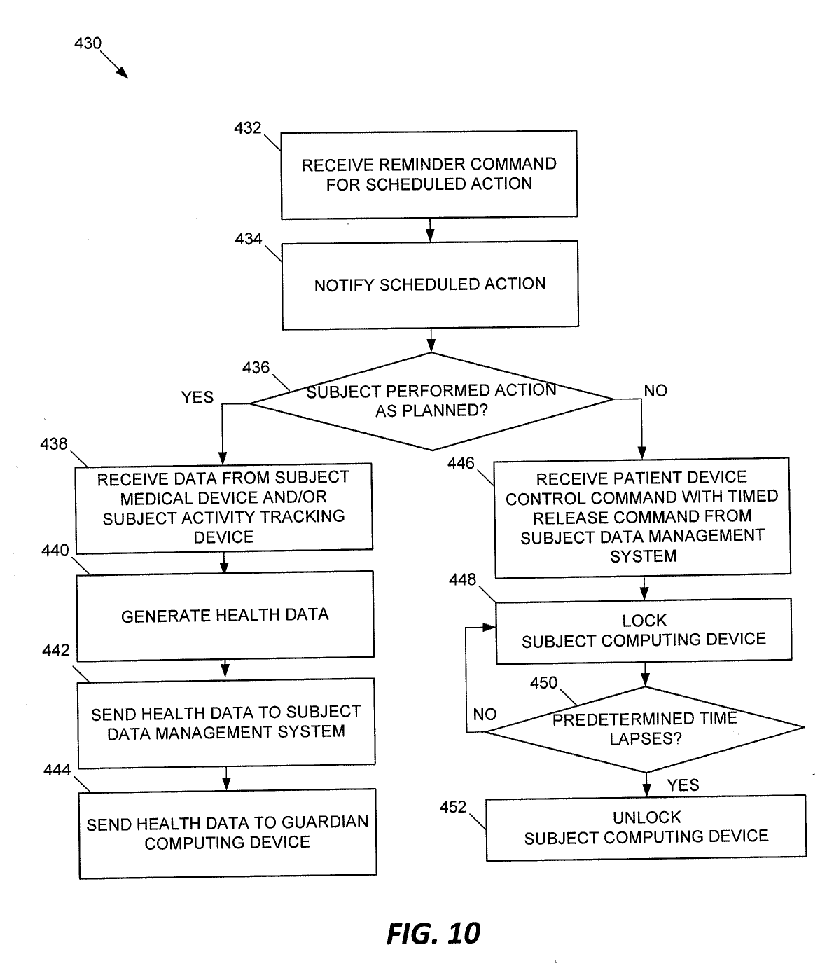

The device control engine 360 operates to receive a subject device control command 186 (FIG. 6) and control access to the subject computing device 104 based on the subject device control command 186. As described herein, the subject device control command 186 can be transmitted to the subject computing device 104 when a scheduled action is not performed by the subject S at a predetermined time as planned in the treatment regimen 122. The subject device control command 186 is then used to lock at least one function of the subject computing device 104. An example method of locking the subject computing device 104 based on the subject device control command 186 is illustrated and described in more detail with reference to FIGS. 9-10.

Although it is primarily described in this disclosure that the subject computing device 104, the subject medical device 102, and the subject activity tracking device 340 are stand-alone devices, it is also possible that at least two of the devices 102, 104, and 340 are combined for simpler use. For example, the subject activity tracking device 340 can be built in either of the subject medical device 102 and the subject computing device 104. Other configurations are also possible in other examples.

FIG. 8 is a block diagram that illustrates example subject health data 370 that are collected and generated by the subject computing device 104. As described above, the subject computing device 104 can generate the subject health data 370 based on the measurement data 118, the activity data 342, and/or other data obtained by the subject computing device 104 or other devices. In some embodiments, the subject health data 370 include subject activity data 372, subject biographical data 374, subject geographical data 376, subject dietary data, atmospheric condition data 378, subject medical measurement data 380, and treatment regimen compliance data 382.

The subject activity data 372 include information about the subject's activities. The subject activity data 372 can be generated based on the activity data 342 transmitted from the subject activity tracking device 340.

The subject biographical data 374 include the subject's biographical information, such as name, address, date of birth, contact, gender, race, ethnicity, and language of preference.

The subject geographical data 376 include information about the location of the subject S. Some embodiments of the subject geographical data 376 are collected by the subject computing device 104 having the location identification device 248, as described in FIG. 5. In some embodiments, the subject geographical data 376 and/or the subject biographical data 374 are used to evaluate or predict the subject's life styles, standards of living, habits, and other factors that can contribute to the subject's health and medical conditions.

The atmospheric condition data 378 include information about the weather at a location where the subject S is present. The atmospheric condition data 378 can include temperature data, precipitation data, wind data, humidity data, dew point data, pressure data, UV index data, visibility data, pollen level data, and air pollution level data. In some embodiments, the atmospheric condition data 378 include weather patterns.

The subject medical measurement data 380 include information about the measurements (e.g., blood glucose readings) taken by the subject S using the subject medical device 102. The subject medical measurement data 380 can be generated based on the measurement data 118 transmitted from the subject medical device 102.

The treatment regimen compliance data 382 include information about whether the subject S has complied with the treatment regimen 122. For example, the treatment regimen compliance data 382 can include information that represents whether the subject S has performed a scheduled action at a predetermined time as planned in the treatment regimen 122, how often the subject S has, or has not, performed the scheduled actions on time, and how early or late the subject S has performed the scheduled actions. Other information can be included in the treatment regimen compliance data 382 in other embodiments.

As such, the subject health data 370 include various factors that can contribute to the subject's chronic disease, thereby allowing objective analysis.

FIG. 9 is a flowchart that illustrates an example method 400 for operating the subject computing device 104.

In this example, at operation 402, the subject computing device 104 receives a reminder command 304 from the subject data management system 108. The reminder command 304 is configured to remind the subject S about a scheduled action that is required in the treatment regimen 122.

At operation 404, the subject computing device 104 operates to notify the scheduled action to the subject S. In some embodiments, the subject computing device 104 generates and presents a notification thereon. The notification can be of various types, such as texts, graphics, sounds, and/or vibrations.

At operation 406, the subject computing device 104 determines whether the subject S has performed the scheduled action as planned in the treatment regimen 122. For example, the subject computing device 104 determines whether the subject S has obtained his or her blood glucose reading using the subject medical device 102 (e.g., a blood glucose meter) at a time predetermined by the treatment regimen 122. If it is determined that the scheduled action has been performed as planned (“YES” at the operation 406), the method 400 moves on to operation 408 and/or alerts the incentive provider 112 to provide rewards or points to the subject computing device as shown in FIG. 1. If not (“NO” at the operation 406), the method 400 continues at operation 416.

At operation 408, the subject computing device 104 receives data (e.g., subject data, such as the measurement data 118 and the subject activity data 342) from the subject medical device 102 and/or the subject activity tracking device 340. In some embodiments, such data include the measurement data 118 and the activity data 342.

At operation 410, the subject computing device 104 generates the health data 370 based on the data (including the measurement data 118 and the activity data 342) received from the subject medical device 102 and/or the subject activity tracking device 340. In addition, the health data 370 can also refer to data independently obtained by the subject computing device 104. Some embodiments of the subject computing device 104 can then store the health data 370 therein.

At operation 412, the subject computing device 104 sends the health data 370 to the subject data management system 108 for further processing.

At operation 414, the subject computing device 104 can send at least part of the health data 370 to the guardian computing device 110 so that the guardian G also monitors the subject's health condition.

If it is determined that the scheduled action has not been performed as planned (“NO” at the operation 406), the operation 416 is performed in which the subject computing device 104 can receive a subject device control command 186 from the subject data management system 108. The subject device control command 186 is configured to lock at least one function of the subject computing device 104.

At operation 418, the subject computing device 104 executes the subject device control command 186 to lock at least one function of the subject computing device 104. The extent to which the subject computing device 104 is locked depends on the subject device control command 186. In some embodiments, the device control command 186 is configured to disable access to all functions of the subject computing device 104 except for at least one of an emergency call option, a call to the healthcare service provider 106 (e.g., the healthcare practitioner P), and the guardian G.

At operation 420, the subject computing device 104 determines whether the subject computing device 104 has received a device release command 188 (FIG. 6) from the subject data management system 108. The device release command 188 is configured to enable the subject computing device 104 to unlock the locked functions of the subject computing device 104. In some embodiments, the device release command 188 is configured independently from the subject device control command 186 and separately transmitted from the subject data management system 108. In other embodiments, the device release command 188 is included in the subject device control command 186, as described in FIG. 10.

Some embodiments of the device release command 188 are configured to unlock the locked functions of the subject computing device 104 after a predetermined release time. The predetermined release time can be preset as a time that lapses after the subject S is denied access to the subject computing device 104. The predetermined release time can be determined by the healthcare service provider 106 and customized for the subject S and/or can be increased due to multiple occurrences of failure to take action or decreased due to an increase in occurrence of taking action at a predetermined time.

Other embodiments of the device release command 188 is configured to unlock the locked functions of the subject computing device 104 after the scheduled action is performed by the subject S, or after one or more other actions are performed by the subject S, the guardian G, the healthcare service provider 106, and/or the healthcare practitioner P.

If it is determined that the device release command 188 has been received (“YES” at the operation 420), the method 400 moves on to operation 422. Otherwise (“NO” at the operation 420), the method 400 returns to the operation 418, at which the subject computing device 104 remains unlocked.

At operation 422, the subject computing device 104 executes the device release command 188 and unlocks the locked functions of the subject computing device 104.

FIG. 10 is a flowchart that illustrates another example method 430 for operating the subject computing device 104.

In this example, operations 432, 434, 436, 438, 440, 442, and 444 are the same as, or similar to, the operations 402, 404, 406, 408, 410, 412, and 414 in FIG. 9. For brevity purposes, the description of the operations 432, 434, 436, 438, 440, 442, and 444 are omitted.

At operation 446, the subject computing device 104 receives a subject device control command 186 from the subject data management system 108. In this example, the subject device control command 186 includes a timed release command 190 configured to unlock the subject computing device 104 once a predetermined release time lapses. The predetermined release time can be preset as a time that lapses after the subject S is denied access to the subject computing device 104. The predetermined release time can be determined by the healthcare service provider 106 and customized for the subject S and/or can be increased due to multiple occurrences of failure to take action or decreased due to an increase in occurrence of taking action at a predetermined time.

Using such a timed release command 190 is advantageous in situations where the subject computing device 104 becomes unconnected to the network 114 after receiving the subject device control command 186. For example, the subject S can move to a location at which the subject computing device 104 is not able to communicate with the subject data management system 108 through the network 114 (e.g., when the subject S moves to a place out of cellular coverage for the subject's mobile device). In such cases, the server based operation of the subject computing device 104 does not work, and the subject computing device 104 cannot receive a release or unlock command from the subject data management system 108. To address this issue, the timed released command 190 can be included in the subject device control command 186 and is used to unlock the subject computing device 104 without need of communicating with the subject management system 108.

At operation 448, the subject computing device 104 executes the subject device control command 186 to lock at least one function of the subject computing device 104. The extent to which the subject computing device 104 is locked depends on the subject device control command 186. In some embodiments, the device control command 186 is configured to disable access to all functions of the subject computing device 104 except for at least one of an emergency call option, a call to the healthcare service provider 106 (e.g., the healthcare practitioner P), and/or the guardian G.

At operation 450, the subject computing device 104 determines whether the predetermined release time (i.e., a time predetermined by the timed release command 190) has lapsed. If it is determined that the predetermined release time has lapsed (“YES” at the operation 450), the method 430 moves on to operation 452. Otherwise (“NO” at the operation 450), the method 430 returns to the operation 448, at which the subject computing device 104 remains unlocked.

At operation 452, the subject computing device 104 executes the timed release command 190 and unlocks the locked functions of the subject computing device 104.

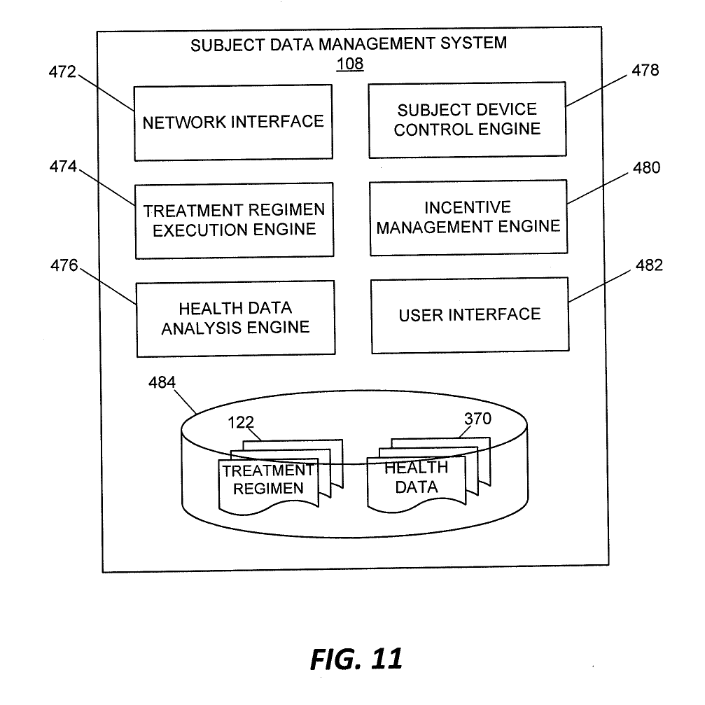

FIG. 11 is a block diagram that illustrates an example of the subject data management system 108. In this example, the subject data management system 108 includes a network interface 472, a treatment regimen execution engine 474, a health data analysis engine 476, a subject device control engine 478, an incentive management engine 480, a user interface 482, and a data store 484.

The network interface 472 is an interface that operates to communicate with other computing devices through the network 114. In some embodiments, the network interface 472 is configured similarly to the wireless network interface 246 as described in FIG. 5.

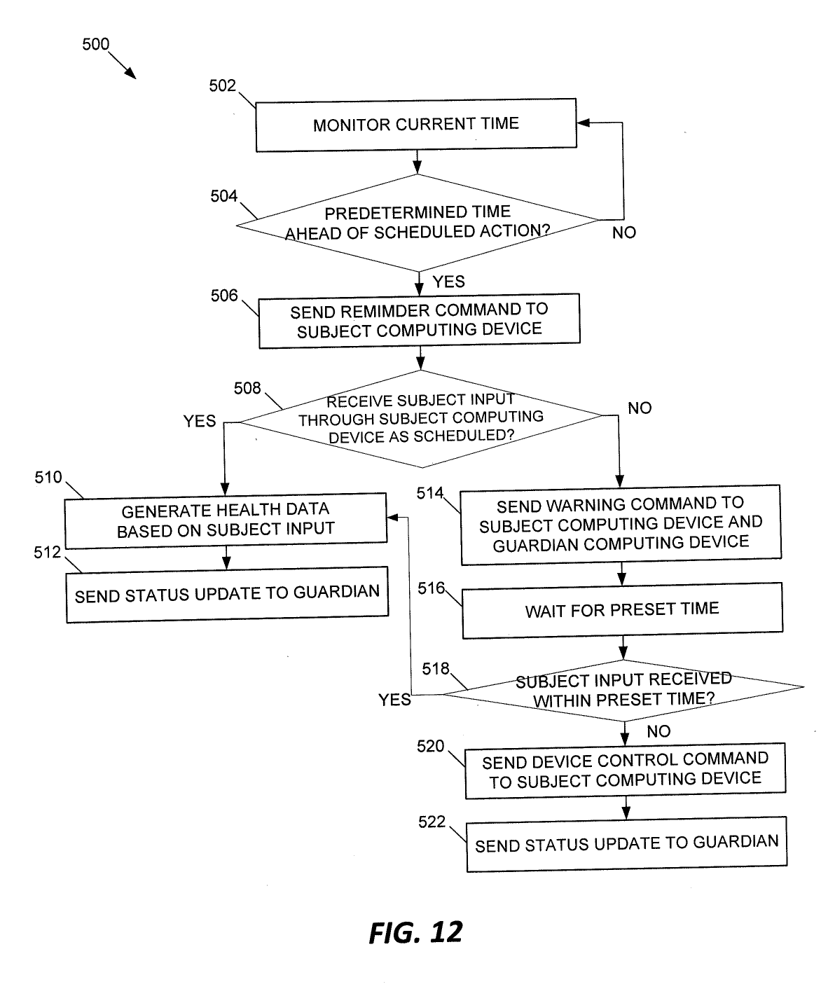

The treatment regimen execution engine 474 operates to execute the treatment regimen 122 for the subject S, as described herein. An example operation of the treatment regimen execution engine 474 is described in more detail with reference to FIG. 12.

The health data analysis engine 476 operates to analyze the health data 370 transmitted from the subject computing device 104, as described herein. An example operation of the health data analysis engine 476 is described in more detail with reference to FIG. 15.

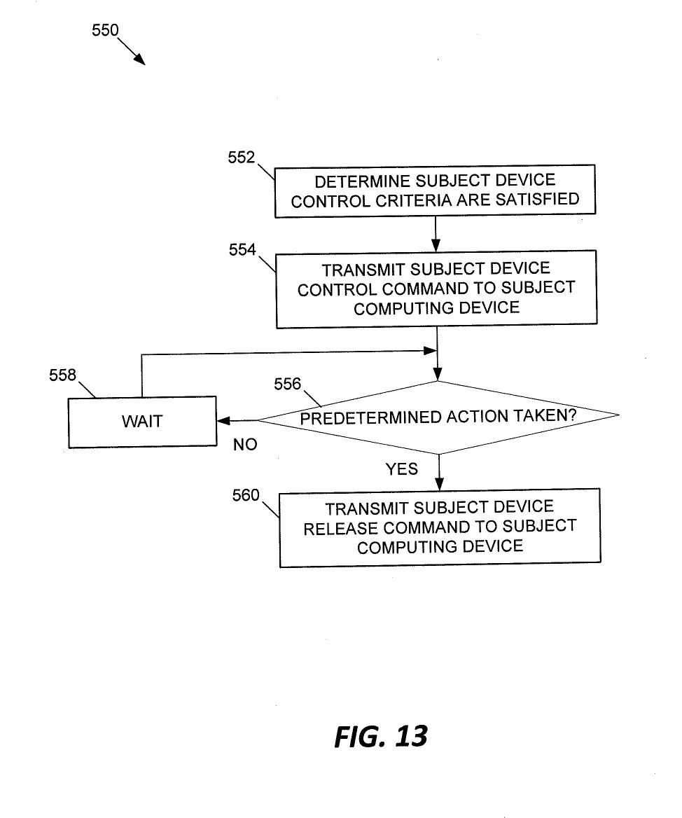

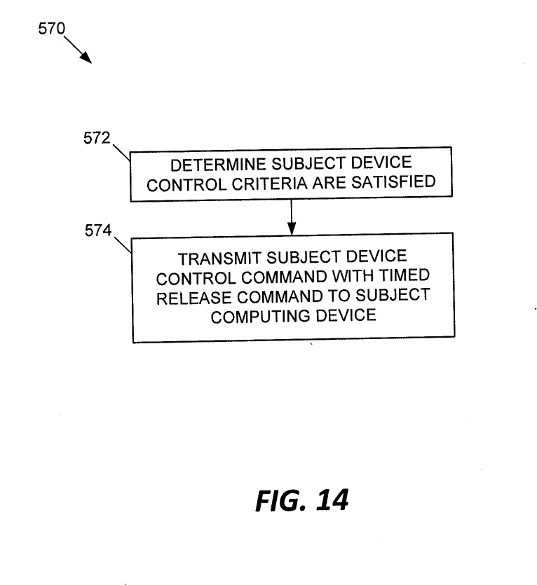

The subject device control engine 478 operates to control the subject computing device 104. In some embodiments, the subject device control engine 478 operates to restrict the subject's access to the subject computing device 104 by transmitting the subject device control command 186 to the subject computing device 104. An example operation of the subject device control engine 478 is described in more detail with reference to FIGS. 13-14.

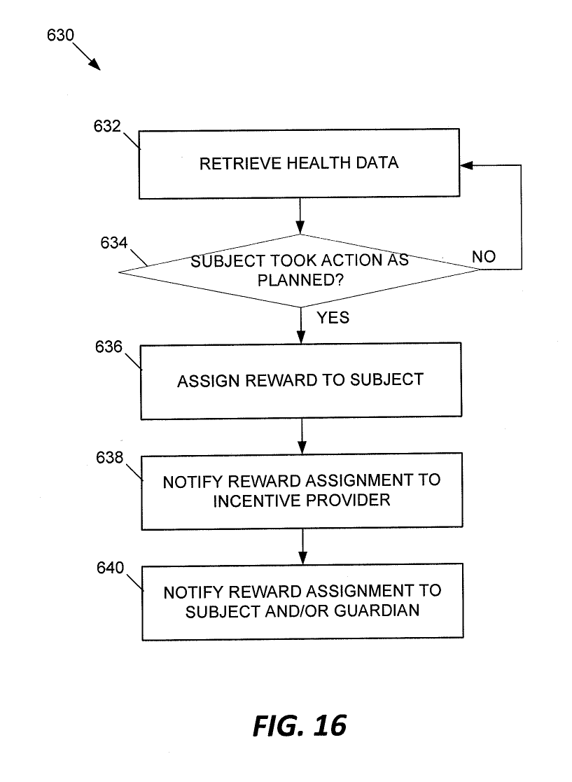

The incentive management engine 480 operates to implement the incentive device 184, as described herein. An example operation of the incentive management engine 480 is described in more detail with reference to FIG. 16.

The user interface 482 provides an interface for a user, such as operators or health practitioners P, to interact with the subject data management system 108. For example, the user interface 482 provides a graphic user interface displayed on a screen of a computing device associated with the subject data management system 108 so that an operator, a healthcare practitioner P, or any other user can see the information displayed thereon, navigate various menu or options, and enter various inputs to the subject data management system 108.

Some embodiments of the user interface 482 are provided as a web portal, such as web service, which is configured to allow programmatic access to data using standard Internet protocols, such as HyperText Transfer Protocol (HTTP).

The data store 484 is configured to store various data that are to be used by the engines of the subject data management system 108. In some embodiments, the data store 484 stores the treatment regimens 122 for different subjects S and the health data 370 for different subjects S.

FIG. 12 is a flowchart that illustrates an example method 500 of operating the treatment regimen execution engine 474. As described herein, the treatment regimen execution engine 474 is configured to implement the treatment regimen 122 customized for the subject S.

In this example, at operation 502, the treatment regimen execution engine 474 operates to monitor a current time. In some embodiments, the treatment regimen execution engine 474 can use a resource of the subject data management system 108 and/or the subject computing device 104 to monitor the current time.

At operation 504, the treatment regimen execution engine 474 operates to determine whether the current time reaches a predetermined time ahead of the scheduled action required by the treatment regimen 122. Such a predetermined time can be customized based on various factors. In some examples, the predetermined time can be set as 1, 5, or 10 minutes ahead of a time at which an action required by the treatment regimen 122 is scheduled to be performed by the subject S. Other examples of the predetermined time can also possible.

If it is determined that the predetermined time is reached (“YES” at the operation 504), the method 500 moves on to operation 506. Otherwise (“NO” at the operation 504), the method 500 returns to the operation 502, at which the treatment regimen execution engine 474 continues to monitor the current time.

At operation 506, the treatment regimen execution engine 474 operates to send a reminder command 304 to the subject computing device 104. As described herein, the subject computing device 104 executes the reminder command 304 and notifies the subject S that a scheduled time is approaching for taking an action required by the treatment regimen 122.

Although it is illustrated in this example that the treatment regimen execution engine 474 is configured to send a single reminder command 304 before the time for the scheduled action, it is also possible in other embodiments that the treatment regimen execution engine 474 operates to transmit a plurality of reminder commands 304 to the subject computing device 104 before the time for the schedule action.

At operation 508, the treatment regimen execution engine 474 determines whether a subject input has been received through the subject computing device 104 as scheduled by the treatment regimen 122. In some embodiments, such a subject input can include the measurement data 118 generated by the subject medical device 102 and transmitted to the subject computing device 104.

If it is determined that the subject input has been received (“YES” at the operation 508), the method 500 moves on to operation 510. Otherwise (“NO” at the operation 508), the method 500 moves on to operation 514.

At operation 510, the treatment regimen execution engine 474 operates to generate the health data 370 based on the subject input.

At operation 512, the treatment regimen execution engine 474 operates to send a status update data to the guardian computing device 110. In some embodiments, the status update data include information about the health data 370. In other embodiments, the status update data include information about whether the subject S has performed a scheduled action as planned in the treatment regimen 122.

If it is not determined that the subject input has been received on time (“NO” at the operation 508), the method 500 continues at the operation 514, at which the treatment regimen execution engine 474 transmits a warning command 322 to the subject computing device 104 and/or the guardian computing device 110, as described herein.

At operation 516, the treatment regimen execution engine 474 then waits for a preset waiting time to see if the subject S will perform the planned action within the preset waiting time after the originally scheduled time.

At operation 518, the treatment regimen execution engine 474 operates to determine whether a subject input has been received through the subject computing device 104 within the preset waiting time. If it is determined that the subject input is received within the waiting time (“YES” at the operation 518), the method 500 moves on to the operation 510. Otherwise (“NO” at the operation 518), the method 500 continues at operation 520.

At operation 520, the treatment regimen execution engine 474 sends a subject device control command 186 to the subject computing device 104 to lock down at least one of the functions of the subject computing device 104 so that the subject S cannot access the locked functions of the subject computing device 104. For example, the subject device control command 186 is executed to lock down the primary functions, such as telephone call, Internet access, and mobile apps, of the subject computing device 104. In some embodiments, the subject computing device 104 can be entirely lock down except for an emergency call, a call to the guardian G, receiving calls, and/or a function of receiving a subject input as planned in the treatment regimen 122.

At operation 522, the treatment regimen execution engine 474 sends a status update data to the guardian computing device 110. In some embodiments, the status update data include information that the subject S has not performed the scheduled action and/or that the subject computing device 104 has been at least partially locked due to the noncompliance.

FIG. 13 is a flowchart illustrating an example method 550 of operating the subject device control engine 478. As described herein, the subject device control engine 478 operates to selectively lock down at least one function of the subject computing device 104.

At operation 552, the subject device control engine 478 operates to determine whether subject device control criteria are satisfied (e.g., whether the subject S has complied with the treatment regimen 122). In some embodiments, the subject device control criteria are met if the subject S does not take an action required by the treatment regimen 122 at a scheduled time, such as measuring a blood glucose level using a blood glucose meter at a predetermined time.

At operation 554, the subject device control engine 478 operates to transmit a subject device control command 186 to the subject computing device 104 if the subject device control criteria have been satisfied at the operation 552. Upon receiving the subject device control command 186, the subject computing device 104 has at least one of its functions locked down to deny the subject's access thereto.

In some embodiments, the subject device control engine 478 is configured to continue to transmit the subject device control command 186 until the subject computing device 104 has actually received the command 186. For example, if the subject device control command 186 is transmitted (or tries to be transmitted) to the subject computing device 104 that is out of communication, the subject device control engine 478 can continue to try sending the subject device control command 186 to the subject computing device 104 until the subject computing device 104 returns back to the communication range and successfully receives the subject device control command 186. In some examples, upon receiving the subject device control command 186, the subject computing device 104 can send a response back to the subject data management system 108 to confirm the receipt of the subject device control command 186.

At operation 556, the subject device control engine 478 determines whether a predetermined action has performed after the subject computing device 104 is locked down. In some embodiments, such a predetermined action is the action that the subject S was supposed to perform on time in accordance with the treatment regimen 122. Examples of the predetermined action include measuring a blood glucose level using a blood glucose meter, injecting insulin using an insulin injection device, replacing an infusion set in an artificial pancreas, taking medications, consuming certain food or drink, performing certain activities, and any other actions scheduled according to the treatment regimen 122. In other embodiments, the predetermined action can be other actions that are to be performed by the subject S (e.g., calling to the guardian G), by the guardian G (e.g., calling to the subject S), or by any other party.

If it is determined that the predetermined action has been taken (“YES” at the operation 556), the method 550 moves on to operation 560. Otherwise (“NO” at the operation 556), the subject device control engine 478 waits until the predetermined action is taken and remains locked.

At operation 560, the subject device control engine 478 operates to transmit a subject device release command 188 to the subject computing device 104 to release the locked functions of the subject computing device 104.

FIG. 14 is a flowchart illustrating another example method 570 of operating the subject device control engine 478. In this example, the method 570 begins at operation 572, which is similar to the operation 552 in FIG. 13. Thus, the description of the operation 572 is omitted for brevity purposes.

At operation 574, the subject device control engine 478 transmits a subject device control command 186 to the subject computing device 104. In this example, the subject device control command 186 includes a timed release command 190 configured to automatically unlock the subject computing device 104 once a predetermined release time lapses. The details of the timed release command 190 are described herein.

Other than the timed release command 190, the operation 574 is similar to the operation 554 in FIG. 13. Thus, the description of the operation 574 is omitted for brevity purposes.

FIG. 15 is a flowchart that illustrates an example method 600 of operating the health data analysis engine 476. As described herein, the health data analysis engine 476 operates to analyze the health data 370 transmitted from the subject computing device 104.

At operation 602, the health data analysis engine 476 retrieves the health data 370 from the data store 484. In some embodiments, the health data analysis engine retrieves the health data 370 for a plurality of subjects S.

At operation 604, the health data analysis engine 476 analyzes the health data 370. In some embodiments, the health data analysis engine 476 performs trend analysis, which compares the health data 370 over time to identify any consistent results. Such trend analysis can be used to understand how the subject S has complied with the treatment regimen 122 and how the subject S has maintained the conditions (e.g., a blood glucose level or insulin level) within a normal range. In some embodiments, the health data analysis engine 476 is configured to determine one or more unusual pattern out of the trend identified for the subject S.

The health data analysis engine 476 can evaluate trends for a variable period of time out of the health data 370. For example, three-day to 14-day trends with diabetes can be monitored and analyzed from three-month collection of the health data 370. The period of time for such trend analysis can be selected in a rolling window basis. Other variations are also possible in other examples.

Some embodiments of the health data analysis engine 476 operate to provide recommendations on the treatment regimen 122 or modification to the treatment regimen based on the analysis of the health data 370. Such recommendations are personalized for each subject S.

At operation 606, the health data analysis engine 476 generates a subject health data report 120. The subject health data report 120 is configured to assist a healthcare practitioner P to provider a healthcare service to the subject S. Some embodiments of the subject health data report 120 include information customized for the subject S. For example, the subject health data report 120 can include summaries of the health data 370, information about health conditions (e.g., statement of patterns), diagnosis of diseases, and/or recommendations or prescriptions for the subject S.

In some embodiments, the subject health data report 120 can be generated periodically. In other embodiments, the subject health data report 120 can be made on demand.

At operation 608, the health data analysis engine 476 generates a subject care message 121. In some embodiments, the subject care message 121 is configured to be transmitted to the subject computing device 104 and can include information about the subject's status and conditions, and recommendations to improve them. For example, the subject care message 121 can include information about whether the subject S has performed an action as planned in the treatment regimen 122 (e.g., measuring a blood glucose level, taking medication or food on time, and/or doing scheduled activities); where the subject S is currently located or has been located; the conditions of the place where the subject S is located (e.g., weather conditions); what the subject S is doing and has been done (e.g., activities); how the subject S is doing in terms of the conditions (e.g., blood glucose levels, or insulin levels); and/or any other information relevant to the subject's health and medical status and conditions. The subject care message 121 can also include recommendations for the subject S, such as what action the subject S should take now (e.g., taking medications, consuming certain food or water, or performing certain activities). The subject care message 121 can include other information in other embodiments. The subject care message 121 can be display on the subject computing device 104.

At operation 610, the health data analysis engine 476 generates a subject status message 123. In some embodiments, the subject status message 123 is configured to be transmitted to the guardian computing device 110 and can include information about the subject's status and conditions. For example, the subject status message 123 can include information similar to those in the subject care message 121. The subject status message 123 can further include recommendations for the guardian G to help or take care of the subject S. The subject status message 123 can include other information in other embodiments. The subject status message 123 can be display on the guardian computing device 110. Upon receiving the subject status message 123 through the guardian computing device 110, the guardian G can be updated with the subject's status and conditions and take any actions, such as making a call to the subject S.

As such, the health data analysis engine 476 can make it easy to monitor a plurality of subjects S and analyze the data associated with the subjects S. Further, the health data analysis engine 476 provides an augmented decision making tool to the healthcare service provider 106, thereby improving effectiveness in caring for the subjects S.

FIG. 16 is a flowchart that illustrates an example method 630 of operating the incentive management engine 480. As described herein, the incentive management engine 480 operates to implement the incentive device 184.

In this example, the method 630 begins at operation 632, at which the incentive management engine 480 retrieves the health data 370 from the data store 484.

At operation 634, the incentive management engine 480 determines whether the subject S has performed the action as required in the treatment regimen 122. In some embodiments, the incentive management engine 480 can look up the treatment regimen compliance data 382 of the health data 370, as described in FIG. 8.

If it is determined that the subject S has taken the required action (“YES” at the operation 634), the method 630 moves on to operation 636. Otherwise (“NO” at the operation 634), the method 630 returns to the operation 632 and retrieves an updated health data 370 if any.

At operation 636, the incentive management engine 480 assigns a reward to the subject S if the subject S has complied with the treatment regimen 122. A reward can be assigned to the subject S based on a predetermined rule. Such a rule can be set up by the healthcare service provider 106, by the incentive provider 112, or by other parties. In some examples, the rule determines when the subject earns a reward, how much reward the subject can earn, and what type of reward the subject can earn. The rule can set up other criteria for assigning rewards to the subject S such as increasing the value of the reward as more compliance is observed or decreasing the value of the reward if less compliance is observed.

The reward can be of various types. Examples of the reward include reward points, awards, coupons, stamps, loyalty programs, cash value that the subject S cash in for, and other suitable types of reward.

The assignment of rewards can be included in the subject health data 370 and stored in the data store 484. In other embodiments, the assignment of rewards can be stored as a data file separately from the subject health data 370.

At operation 638, the incentive management engine 480 operates to notify the reward assignment to the incentive provider 112. Once the incentive provider 112 is informed of the fact that a certain amount of rewards have been assigned to the subject S, the incentive provider 112 can provide goods and/or services to the subject S in return of the rewards assigned to the subject S. In some embodiments, the incentive management engine 480 provides a reminder to the subject S periodically (such as daily, weekly, or monthly update). Such a reminder can include various pieces of information regarding the reward assignment, such as the total amount of reward assigned to the subject S, the log of reward assignments for a predetermined period of time, and instruction for using the rewards.

At operation 640, the incentive management engine 480 operates to notify the reward assignment to the subject S and/or the guardian G so that the subject S and/or the guardian G become aware that the subject S has earned the rewards and can utilize them with the incentive provider 112.

As described herein, the systems and method in accordance with the present disclosure are programmed to lock functionality of a subject's device (e.g., a smartphone) if the subject does not comply with a treatment regimen. In addition, the systems and method is also configured to deliver incentives if the subject complies with the treatment regimen. Such disincentives and incentives can be particularly effective for children or teenagers because they value the functionality of their devices. For different subjects, the disincentives and incentives can be modified for improved results. For example, regarding disincentives, different functions of a subject's device can be selectively locked upon non-compliance with the treatment regimen, depending on the subject's age. As such, the disincentive and incentive programs as described herein can modify the subject's behavior and encourage the subject to comply with a treatment regimen.

Various data inputs can be used to determine whether the subject has complied with a predetermined treatment regimen. As described herein, a subject medical device, such as a glucose meter, is used to generate measurement data which can be used for such determination. In other examples, a software application, such as a mobile app, which runs on the subject's device, can be used to generate data usable for verifying the subject's compliance.

Although the disclosure has been described herein using exemplary techniques, components, and/or processes for implementing the systems and methods of the present disclosure, it should be understood by those skilled in the art that other techniques, components, and/or processes or other combinations and sequences of the techniques, components, and/or processes described herein may be used or performed that achieve the same functions and/or results described therein and which are included within the scope of the present disclosure.

The various examples and teachings described above are provided by way of illustration only and should not be construed to limit the scope of the present disclosure. Those skilled in the art will readily recognize various modifications and changes that may be made without following the examples and applications illustrated and described herein, and without departing from the true spirit and scope of the present disclosure.

What is claimed is:

1. A method of modifying compliance with a treatment regimen by a subject having a chronic disease, the method comprising:
obtaining a treatment regimen configured to manage the chronic disease, the treatment regimen including at least one action required to be performed by the subject using a subject computing device at one or more predetermined times;
determining, using at least one computing device, that the at least one action is performed by the subject at the one or more predetermined times; and
if the at least one action is not performed by the subject at the one or more predetermined times, transmitting, using the at least one computing device, a subject device control command to the subject computing device, the subject device control command configured to lock at least one function of the subject computing device for a predetermined release time or until the at least one action is performed by the subject.

2. The method of claim 1, wherein the subject device control command is configured to disable access to all functions of the subject computing device except for an emergency call, a call to a health care provider, a call to a parent or guardian, or combinations thereof.

3. The method of claim 1, further comprising:
transmitting a subject device release command to the subject computing device, the subject device release command configured to unlock the at least one locked function of the subject computing device after the predetermined release time or after the at least one action is performed by the subject.

4. The method of claim 1, wherein the device control command includes a scheduled release command configured to unlock the at least one locked function of the subject computing device after a predetermined release time.

5. The method of claim 1, further comprising:
transmitting a reminder command to the subject computing device before the one or more predetermined times, the reminder command configured to enable the subject computing device to generate at least one notification of the at least one action to the subject ahead of the one or more predetermined times.

6. The method of claim 1, further comprising:
transmitting status data to a guardian of the subject, the status data including information representing that the at least one action is not performed at the one or more predetermined times.

7. The method of claim 1, further comprising:
assigning incentive information to the subject if the at least one action is performed by the subject at the one or more predetermined times.

8. The method of claim 7, further comprising:
notifying the assignment to an incentive provider, the incentive provider providing a reward program for the subject.

9. The method of claim 1, further comprising:
receiving subject data associated with the at least one action from the subject computing device;
generating subject health data based on the subject data;
analyzing the subject health data; and
generating a subject health data report based on the analysis of the subject health data, the subject health data report including information customized for the subject.

10. The method of claim 9, further comprising:
sending the subject health data report to a healthcare practitioner, the subject health data report configured to assist the healthcare practitioner to modify the treatment regimen or provide a healthcare service to the subject.

11. The method of claim 1, wherein the treatment regimen is determined based on medical and health conditions of the subject.

12. The method of claim 1, wherein the at least one action is a blood glucose test using a blood glucose meter, the blood glucose meter configured to be connected to the subject computing device to transmit measurements from the blood glucose meter to the subject computing device.

13. The method of claim 1, further comprising:
receiving at least one of subject activity data, subject biographical data, subject geographical data, and atmospheric data from the subject computing device; and
generating subject health data based on the at least one of the data.

14. A system for modifying compliance with a treatment regimen for a subject with a chronic disease, the system comprising:
at least one processing device configured to control operation of the system;
one or more computer readable data storage media storing software instructions that, when executed by the at least one processing device, cause the system to:
obtain a subject treatment regimen configured to manage the chronic disease, the subject treatment regimen including at least one action required to be performed by the subject using a subject's computing device at one or more predetermined times;
determine that the at least one action is performed by the subject at the one or more predetermined times; and
if the at least one action is not performed by the subject at the one or more predetermined times, transmit a subject device control command to the subject computing device, the subject device control command configured to lock at least one function of the subject computing device for a predetermined release time or until the subject performs that at least one action.

15. The system of claim 1, wherein the subject device control command is configured to disable access to all functions of the subject computing device except for an emergency call, a call to a health care provider, a call to a parent or guardian, or combinations thereof.

16. The system of claim 14, wherein the software instructions further cause the system to:
transmit a subject device release command to the subject computing device, the subject device release command configured to unlock the at least one locked function of the subject computing device after the predetermined release time or after the at least one action is performed by the subject.

17. The system of claim 14, wherein the device control command includes a scheduled release command configured to unlock the at least one locked function of the subject computing device after a predetermined release time.

18. The system of claim 14, wherein the software instructions further cause the system to:
transmit a reminder command to the subject computing device before the one or more predetermined times, the reminder command configured to enable the subject computing device to generate at least one notification of the at least one action to the subject ahead of the one or more predetermined times.

19. The system of claim 14, wherein the software instructions further cause the system to:
transmit status data to a guardian of the subject, the status data including information representing that the at least one action is not performed at the one or more predetermined times.

20. The system of claim 14, wherein the software instructions further cause the system to:
assign incentive information to the subject if the at least one action is performed by the subject at the one or more predetermined times.

21. The system of claim 20, wherein the software instructions further cause the system to:
notify the assignment to an incentive provider, the incentive provider providing a reward program for the subject.

22. The system of claim 14, wherein the software instructions further cause the system to:
receive subject data associated with the at least one action from the subject computing device;
generate subject health data based on the subject data;
analyze the subject health data; and
generate a subject health data report based on the analysis of the subject health data, the subject health data report including information customized for the subject.

23. The system of claim 22, wherein the software instructions further cause the system to:
send the subject health data report to a healthcare practitioner, the subject health data report configured to assist the healthcare practitioner to modify the treatment regimen or provide a healthcare service to the subject.

24. The system of claim 14, wherein the treatment regimen is determined based on medical and health conditions of the subject.

25. The system of claim 14, wherein the at least one action is a blood glucose test using a blood glucose meter, the blood glucose meter configured to be connected to the subject computing device to transmit measurement data from the blood glucose meter to the subject computing device.

26. The system of claim 14, wherein the software instructions further cause the system to:
receive at least one of subject activity data, subject biographical data, subject geographical data, and atmospheric data from the subject computing device; and
generate subject health data based on the at least one of the data.

27. A computer-readable storage medium comprising software instructions that, when executed, cause at least one processing device to:
obtain a subject behavior plan configured to manage the chronic disease, the subject treatment regime including at least one action required to be performed by the subject using a subject computing device at one or more predetermined times;
determine that the at least one action is performed by the subject at the one or more predetermined times; and
if the at least one action is not performed by the subject at the one or more predetermined times, transmit a subject device control command to the subject computing device, the subject device control command configured to lock at least one function of the subject computing device for a predetermined release time or until the subject performs the at least one action.

28. A method of encouraging compliance with a treatment regimen by a subject with a chronic disease using a subject computing device, the method comprising:
generating a notification of an action required by the treatment regimen using the subject computing device, the action required to be performed by the subject with a subject medical device at a predetermined time;
if the action is not performed at the predetermined time, receiving a subject device control command from at least one server computing device, the subject device control command configured to lock at least one function of the subject computing device; and
disabling access to the at least one function of the subject computing device based on the subject device control command.

29. The method of claim 28, wherein disabling access includes disabling access to the at least one function of the subject computing device for a predetermined release time or until the subject performs the at least one action.

30. The method of claim 28, wherein the subject device control command is configured to disable access to all functions of the subject computing device except for an emergency call, a call to a health care provider, a call to a parent or guardian, or combinations thereof.

31. The method of claim 28, further comprising:
receiving a device release command from the at least one server computing device, the device release command configured to unlock the at least one locked function of the subject computing device; and
releasing the disablement of the at least one function of the subject computing device based on the device release command.

32. The method of claim 28, further comprising:
releasing the disablement of the at least one function of the subject computing device based on the subject device control command, the subject device control command including a scheduled release command configured to unlock the at least one disabled function of the subject computing device after a predetermined release time.

33. The method of claim 28, further comprising:
receiving a reminder command from the at least one server computing device before the predetermined time, the reminder command configured to enable the subject computing device to generate the notification of the subject action.

34. The method of claim 28, further comprising:
if the subject action is performed at the predetermined time, transmitting subject data associated with the performed subject action to the at least one server computing device, the subject data including at least one of subject medical measurement data, subject activity data, subject biographical data, subject geographical data, and atmospheric condition data.

35. The method of claim 28, wherein:
the subject medical device includes a blood glucose meter configured to be connected to the subject computing device to transmit measurements from the blood glucose meter to the subject computing device; and
the action is a blood glucose test using the blood glucose meter.
